(19) United States
(12) Patent Application Publication
(10) Pub. No.: US 2021/0373873 A1
(43) Pub. Date: Dec. 2, 2021

(54) MANIFEST AND PAYLOAD DELIVERY

(71) Applicant: Arm Cloud Technology, Inc., San Jose, CA (US)

(72) Inventors: Christopher James Styles, Cambridge (GB); Linlin Gao, Austin, TX (US)

(21) Appl. No.: 16/884,784

(22) Filed: May 27, 2020

(51) Int. Cl. G06F 8/65; G06F 21/45; H04L 9/30; G06F 11/10

(52) U.S. Cl. CPC G06F 8/65; G06F 21/45; G16H 40/40; G06F 11/1004; H04L 9/30

(57) ABSTRACT

A method for delivering an update manifest and an update payload to a target device, the method comprising: receiving, at the target device, security credentials for the target device, the target device being configured to receive the update manifest and the update payload via a remote connection interface using the security credentials; receiving, at the target device, the update manifest from a host device via a local connection interface; and applying, at the target device, the update payload in accordance with the update manifest.

The present techniques generally relate to delivering an update manifest and an update payload to a device. More particularly, the techniques relate to delivering a firmware manifest and a firmware update to a target device.

Cloud, or distributed network, computing services are becoming more common. More and more devices are being connected to the cloud, for example as part of the “Internet of Things” (IoT). For example, relatively small devices such as temperature sensors, healthcare monitors and electronic door locks can be connected to the cloud so that they can be accessed and controlled using remote systems. For example, a door may be remotely opened from a remote platform, or data from a temperature sensor or healthcare monitor may be aggregated at a remote location and accessed from another device. Hence, there is an increasing amount of data being collected by cloud platforms and their providers.

In order to provide a firmware update of a device, a host computer is required to upload a signed firmware manifest to a cloud based device management service and to upload the firmware update to a cloud based file hosting service. The device management service can then trigger a download of the signed firmware manifest to the device before the firmware update is provided from the file hosting service to the device. If network connectivity between the device and the cloud is lost, and the only way to recover the connection is by a firmware update, then the device must be erased, reprogrammed and re-provisioned, which may be time and resource consuming.

It would therefore be desirable to provide an alternative system and method for delivering an update manifest and an update payload to a device, in particular for updating the firmware of a device.

According to a first aspect of the present technique, there is provided a method for delivering an update manifest and an update payload to a target device, the method comprising: receiving, at the target device, security credentials for the target device, the target device being configured to receive the update manifest and the update payload via a remote connection interface using the security credentials; receiving, at the target device, the update manifest from a host device via a local connection interface; and applying, at the target device, the update payload in accordance with the update manifest.

According to a second aspect of the present technique, there is provided a target device comprising: communication circuitry configured to: receive security credentials for the target device; receive an update manifest and an update payload via a remote connection interface using the security credentials; and receive the update manifest from a host device via a local connection interface; and a processor configured to: apply the update payload in accordance with the update manifest.

As will be appreciated by one skilled in the art, the present techniques may be embodied as an apparatus, a system, a method or a computer program. Accordingly, present techniques may take the form of an entirely hardware embodiment, an entirely software embodiment, or an embodiment combining software and hardware aspects.

Embodiments will now be described with reference to the accompanying figures of which:

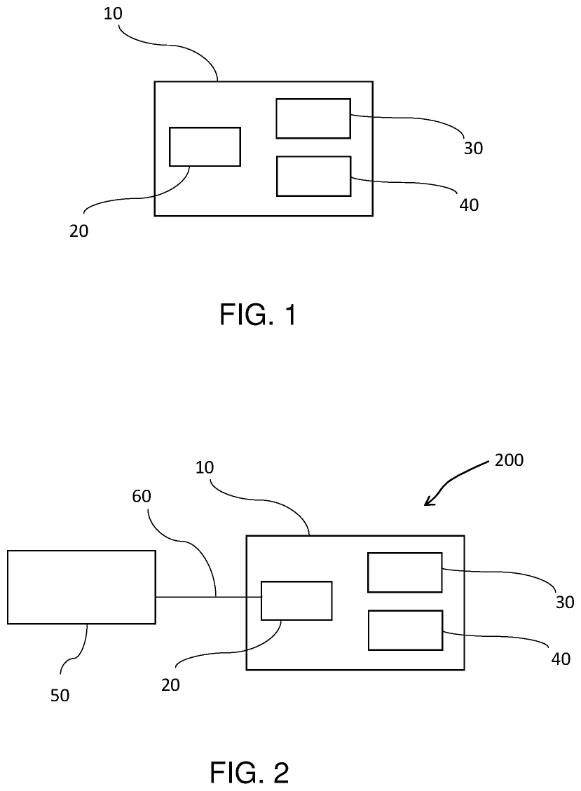

FIG. 1 illustrates a schematic diagram of an apparatus according to various examples;

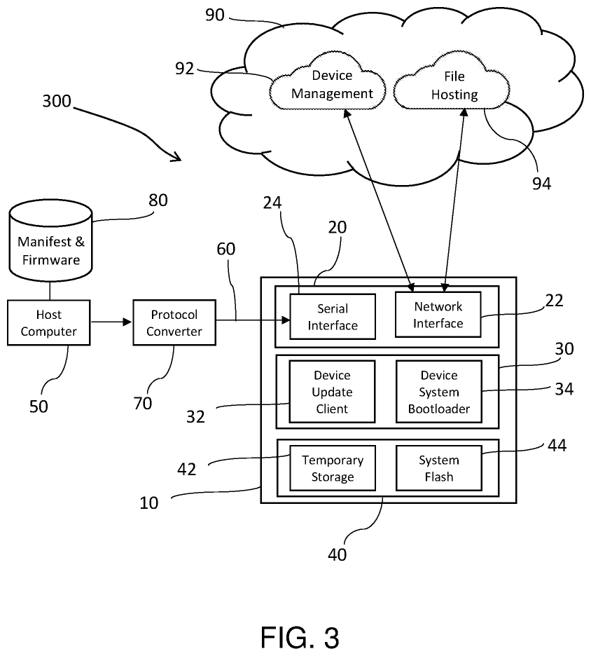

FIG. 2 illustrates a schematic diagram of a system according to various examples;

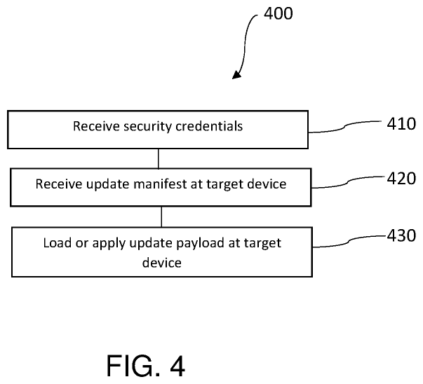

FIG. 3 illustrates a schematic diagram of a system incorporating an apparatus according to various examples;

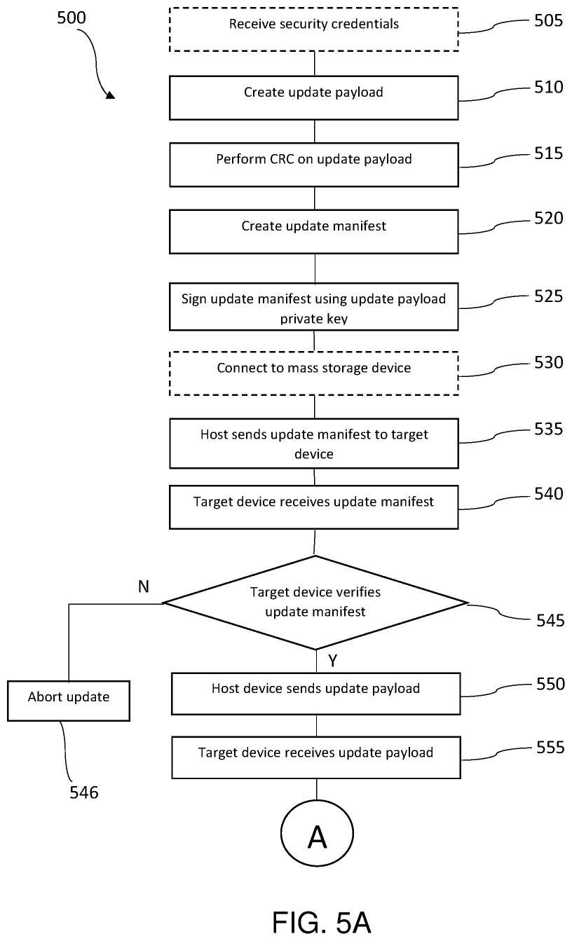

FIG. 4 illustrates a flow diagram of blocks of a method according to various examples; and

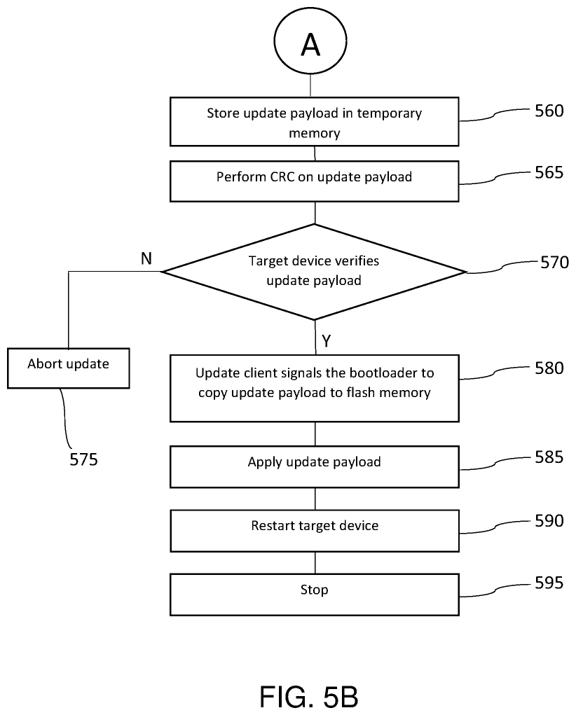

FIGS. 5A and 5B, functionally connected at connection point A, together illustrate a flow diagram of blocks of a method according to various examples.

Reference is made in the following detailed description to the accompanying drawings, wherein like numerals may designate like parts throughout that are corresponding and/or analogous. It is to be understood that other embodiments may be utilized. Furthermore, structural and/or other changes may be made without departing from claimed subject-matter.

The accompanying drawings and following description provide details of the present techniques for delivering an update manifest and an update payload to a device.

FIG. 1 illustrates a schematic diagram of an apparatus 10, where the apparatus 10 may be in the form of a target device 10 to which an update manifest and an update payload is to be delivered. In some embodiments the update manifest may be a firmware manifest and the update payload may be a firmware update or a firmware delta update.

The target device 10 may be considered to be a remote device and may be known as an Internet of Things (IoT) device.

The target device 10 comprises communication circuitry 20 configured to receive security credentials for the target device 10. The security credentials may be received from a provisioning device or server, for example a server which forms part of a distributed, or cloud, computing environment or network 90, as illustrated in FIG. 3.

The security credentials may be used to provision and manage the target device 10 in a distributed computing environment 90, such as a cloud based environment 90. The presently described embodiments allow for the updating of the firmware of the target device 10 using a local connection interface without a full re-provisioning of the target device 10, as the security credentials are maintained at the target device 10 after the firmware update.

The security credentials may include various data relating to device security and identity. The security credentials may for instance comprise a public key, which can be used to authenticate or verify a message or data sent from a sender to the target device 10, where the sent message or data is signed with the sender's private key. The security credentials may comprise identity information for the target device 10. The security credentials may, for example, comprise a firmware update public key for verifying the authenticity of firmware updates, which would be signed with a corresponding, or associated, firmware update private key.

As illustrated in FIG. 3 in more detail, the communication circuitry 20 comprises a remote connection interface 22 and a local connection interface 24. The local connection interface 24 of the communication circuitry 20 may be any interface that is native to the target device 10 that does not require specialized infrastructure. That is, the local connection interface 24 of the communication circuitry 20 may be any interface that does not require, for example, access points, a subscriber identity module (SIM), or connection credentials.

The local connection interface 24 of the communication circuitry 20 may comprise circuitry that provides both input and output functions using one or more supported data or communication channels 60, which may include, for example, an Inter-Integrated Circuit (I²C) serial computer bus, a serial peripheral interface (SPI) bus, a Controller Area Network (CAN) bus, a Local Area Network (LAN), or any other local communication method.

In some embodiments a personal area network, such as a Bluetooth Low Energy (BT-LE) personal area network, may be used as the local communication channel 60, if the target device 10 itself has appropriate personal area network connectivity, for example Bluetooth capability. Other personal area networks, such as ZigBee, may also be used.

In some embodiments, the local connection interface 24 may be a serial interface. The serial interface may have a line speed of 115,200 bits per second, and have a serial port parameter setting of 8-N-1 providing 1 start bit, 8 data bits, no parity bits, and 1 stop bit, however, it will be understood that different line rates and serial port parameter settings may be applied within the scope of other embodiments.

The communication circuitry 20 is configured to receive an update manifest and an update payload via the remote connection interface 22 using the security credentials. The remote connection interface 22 may be connectable to a distributed, or cloud, computing environment 90, which may comprise device management apparatus 92 and file hosting apparatus 94.

The communication circuitry 20 is configured to receive the update manifest from a host device 50 via the local connection interface 24.

As illustrated in FIG. 1, the target device 10 comprises a processor 30 configured to load or apply the update payload in accordance with the update manifest. The processor 30 may comprise processing circuitry. The processor 30 or processing circuitry may be considered to be an update processor or update processing circuitry, and may comprise a device update client 32 and a device system bootloader 34, as illustrated in FIG. 3.

As illustrated in FIG. 1, the target device 10 may comprise memory 40 for storing security credentials. The memory 40 may be in the form of storage circuitry. The memory 40 may comprise non-volatile storage, such as a flash memory, and/or volatile storage, such as a cache memory allowing high-speed data access. The memory 40 may store a public key to be used in a cryptographic system, such as in the verification of a message or data sent from an external sender. The memory 40 may store the update manifest and/or the update payload.

As illustrated in more detail in FIG. 3, the memory 40 may comprise temporary memory or storage 42 and system flash memory 44. The purpose of the temporary memory storage 42 and system flash memory 44 may be as described herein in relation to updating firmware on a target device 10. The memory 40 may also store target device description data, which may comprise one or more of vendor identification, model identification for the respective target device 10 and serial number of the respective target device 10. Such target device description data may provide information to allow identification of firmware for the respective target device 10.

FIG. 2 illustrates a schematic diagram of a system 200 comprising a target device 10 and a host device 50. The host device 50 is connected to the target device 10 via a local connection 60. The host device 50 is configured to transmit, to the target device 10, the update manifest and the update payload.

In some embodiments, the host device 50 may transmit, to the target device 10, a firmware manifest and a firmware update. The target device 10 may receive, from the host device 50, the firmware manifest and the firmware update.

FIG. 3 illustrates a schematic diagram of a system 300 with additional optional components. FIG. 3 also illustrates a target device 10 in more detail.

Between host device 50 and target device 10 there may be a protocol converter 70 to convert from a communication protocol at the host device 50 to a different communication protocol at the target device 10, in order to achieve interoperability. For example conversion from a universal serial bus (USB) file system on the host device 50 to the interface of the target device 10 may be required, for example if the target device 10 uses serial universal asynchronous receiver-transmitter (UART) protocol.

In FIG. 3, the host computer 50 is connected to a mass storage device 80, the mass storage device 80 being configured to store the update payload and corresponding update manifest for the target device 10. In some embodiments the update payload and corresponding update manifest for the target device 10 are created and/or stored at the host device 50.

The method 400 of operation of the apparatus 10 is described in the following paragraphs with reference to FIG. 4, and is described in relation to receiving an update manifest at a target device 10 from a host device 50, and applying an update payload to the target device 10. It will be understood that the steps of the method 400 may be repeated for further updates.

At block 410 security credentials for the target device 10 are received, at the target device 10. The target device 10 may be configured to receive the update manifest and the update payload via a remote connection interface 22 using the security credentials.

At block 420 the update manifest is received, at the target device 10, from a host device 50, via a local connection interface 24. The host device 50 and target device 10 are connected by a local communication channel 60.

At block 430 the update payload is loaded or applied, at the target device 10, in accordance with the update manifest.

The method 500 of operation of the apparatus 10 and in particular operation of the apparatus 10 in the context of a system 300 is described in the following paragraphs with reference to FIGS. 5A and 5B where the reference A in the figures indicates a continuation point between FIG. 5A and FIG. 5B. Specifically, in FIGS. 5A and 5B, the operation of the apparatus 10 and system 300 in which the apparatus operates is described in relation to delivering an update manifest, which may be in the form of a firmware manifest, at a target device 10 from a host device 50, and loading or applying an update payload, which may be in the form of a firmware update, at the target device 10, in accordance with the update manifest.

It will be understood that the steps of the method 500 may be repeated for further updates and that some of the steps of the method may be omitted or performed in a different order to that of the below described example.

At block 505, as part of target device provisioning, security credentials for the target device 10 are received at the target device 10. Such a block may be optional in the method where the target device already has appropriate security credentials.

The target device 10 is configured to receive an update manifest and an update payload via a remote connection interface 22 using the security credentials. The update manifest and update payload may, for example, be received via the remote connection interface 22 from a distributed computing environment 90. The security credentials received at the target device 10 are required to manage the target device 10 via the remote connection interface 22.

The security credentials for the target device 10 comprise a public key, which can be used to authenticate or verify a message or data sent from a sender to the target device 10, where the sent message or data is signed with the sender's private key. Thus by retaining the public key of a key pair, the target device 10 can verify that the sender of the sent message or data had access to the associated private key. This may help to ensure that the message or data has not been tampered with.

If the target device 10 loses connection to the distributed computing environment 90 and requires a update payload, such as a firmware update, in order to restore the connection, or if there are connection issues between the target device 10 and the distributed computing environment 90 that the device cannot diagnose, but which will require an updated payload, such as a firmware update, in order to resolve, then the target device 10 may receive the updated payload without the use of the connection to the distributed computing environment 90 by the use of a local connection interface 24, as will be described in more detail below.

In some examples, the loss of the remote connection to the target device 10, or the incidence of connection issues between the target device 10 and the distributed computing environment 90, can be detected, for example by the lack of communication from the target device 10 to the distributed computing environment 90. Once the loss of the remote connection is detected, a payload update can be subsequently delivered to the target device 10 via the local connection interface 24, over a local communication channel 60.

In other examples the loss of the remote connection to the target device 10 may not be detected, but the local connection interface 24 may be used to provide payload updates.

At block 510 the update payload is created. The update payload is intended to update the target device 10. The update payload may be a software update. More specifically, the update payload may be a firmware update. If the update payload is a firmware update, it may comprise a firmware delta update.

A firmware delta update is a firmware update comprising the minimal changes required to the current firmware to bring the firmware up to date. Knowledge of the state of the current firmware is required in order to be able to compute the delta update, since delta updates only make sense if it is already known what is in the current firmware, such that the use of delta updates may also provide further securing of the target device 10, thereby assisting in avoiding the misappropriation of the target device 10.

Delta updates may be provided to minimize data transfer by defining values required to change the current firmware state to the desired firmware state. The host device 50 may compute a delta update for the firmware to provide the minimum changes required to bring the firmware up to date.

At block 515 a first cyclic redundancy check is performed on the update payload to compute a first check value. The first cyclic redundancy check may be performed at the host device 50.

At block 520 the update manifest is created for the update payload. In the case where the update payload is a software update the update manifest is a software manifest. In the case where the update payload is a firmware update the update manifest is a firmware manifest. The update manifest comprises the first check value from the first cyclic redundancy check.

The update manifest comprises a location identifier for the update payload. In some embodiments, the update manifest may comprise a location identifier for the update payload in the form of a universal asynchronous receiver-transmitter download location for the update payload.

At block 525 the update manifest is signed using a private key before sending to the target device 10. The private key is paired with the public key that the target device 10 received during provisioning.

The host device 50 may then be connected to the target device 10 via a local connection interface 24 at the target device 10. As previously mentioned such the local connection interface 24 may be, for example, a serial interface.

Optionally, at block 530, illustrated by a dashed box, the host device 50 may be connected to a mass storage device 80. The mass storage device 80 may be configured to store the update payload and corresponding update manifest for the target device 10. The update payload and corresponding update manifest may then be provided to the host device 50 for sending to the target device 10. The mass storage device 80 may be attached to or associated with the host device 50.

The mass storage device 80 may be platform independent. Therefore, when the host device 50 is connected to the mass storage device 80, where the mass storage device 80 stores the update payload and the associated update manifest, the update manifest and update payload may be dragged and dropped from the mass storage device 80 connected to the host device 50 to the target device 10 for delivery thereto.

At block 535 the update manifest is sent to the target device 10 from the host device 50 via the local connection interface 24. The location identifier identifies the location of the correct update payload to be received by the target device 10.

At block 540 the update manifest is received, at the target device 10, from the host device 50 via the local connection interface 24.

At block 545 the authenticity of the received update manifest is verified, at the target device 10, by using the public key. The download location for the update payload is received as part of the update manifest. The download location for the update payload may be in the form of a universal asynchronous receiver-transmitter download location for the update payload. The first check value is also received, at the target device 10, from the first cyclic redundancy check as part of the update manifest. If the target device is unable to verify the update manifest, then the update process is aborted at block 546.

At block 550, in response to the verification of the authenticity of the received update manifest, the update payload is sent to the target device 10 from the host device 50 via the local connection interface 24.

At block 555 the update payload for the target device 10 is received, at the target device 10, via the local connection interface 24 from the host device 50.

At block 560 the update payload received at the target device 10 is stored in a temporary memory or storage 42 of the target device 10.

At block 565 a second cyclic redundancy check is performed on the received update payload to compute a second check value. The second cyclic redundancy check is performed at the target device 10.

At block 570 the second check value is compared to the first check value, at the target device 10. Should the second check value not match with the first check value then at block 575 the update is aborted.

If the second check value matches with the first check value, then at block 580 a device update client 32 of the target device 10 provides instruction to a target device system bootloader 34 to copy, or load, the update payload to a target device flash memory 44. As a consequence of the instruction, the update payload is copied from the temporary memory or storage 42 of the target device 10 and loaded into the target device flash memory 44.

At block 585 the update payload is applied, at the target device 10, in accordance with the update manifest.

At block 590, following the copying or loading of the update payload to the target device flash memory 44, the target device 10 is restarted, and the update process ends at block 595.

Following restart of the target device 10 the security credentials at the target device 10 are maintained. Since the security credentials at the target device 10 are maintained there is no need to re-provision the target device 10.

While the above-described embodiments of the disclosed methods relate to providing an update manifest and an update payload to a target device 10 via a local connection interface 24 without the required for re-provisioning, it will be appreciated that the methods described herein can also be used for, or as part of, provisioning or commissioning of a target device 10, for example while in the field, via the local connection interface 24, thereby securely providing appropriate configuration at the point of deployment.

As will be appreciated by one skilled in the art, the present techniques may be embodied as a system, method or computer program product. Accordingly, the present techniques may take the form of an entirely hardware embodiment, an entirely software embodiment, or an embodiment combining software and hardware.

Furthermore, the present techniques may take the form of a computer program product embodied in a computer readable medium having computer readable program code embodied thereon. The computer readable medium may be a computer readable signal medium or a computer readable storage medium. The computer readable storage medium may be a non-transitory computer readable storage medium. A computer readable medium may be, for example, but is not limited to, an electronic, magnetic, optical, electromagnetic, infrared, or semiconductor system, apparatus, or device, or any suitable combination of the foregoing.

Computer program code for carrying out operations of the present techniques may be written in any combination of one or more programming languages, including object oriented programming languages and conventional procedural programming languages.

For example, program code for carrying out operations of the present techniques may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language such as Verilog™ or VHDL (Very high speed integrated circuit Hardware Description Language).

The program code may execute entirely on the user's computer, partly on the user's computer and partly on a remote computer or entirely on the remote computer or server. In the latter scenario, the remote computer may be connected to the user's computer through any type of network. Code components may be embodied as procedures, methods or the like, and may comprise sub-components which may take the form of instructions or sequences of instructions at any of the levels of abstraction, from the direct machine instructions of a native instruction set to high-level compiled or interpreted language constructs.

It will also be clear to one of skill in the art that all or part of a logical method according to the preferred embodiments of the present techniques may suitably be embodied in a logic apparatus comprising logic elements to perform the steps of the method, and that such logic elements may comprise components such as logic gates in, for example a programmable logic array or application-specific integrated circuit. Such a logic arrangement may further be embodied in enabling elements for temporarily or permanently establishing logic structures in such an array or circuit using, for example, a virtual hardware descriptor language, which may be stored and transmitted using fixed or transmittable carrier media.

In one alternative, an embodiment of the present techniques may be realized in the form of a computer implemented method of deploying a service comprising steps of deploying computer program code operable to, when deployed into a computer infrastructure or network and executed thereon, cause said computer system or network to perform all the steps of the method.

In a further alternative, the preferred embodiment of the present techniques may be realized in the form of a data carrier having functional data thereon, said functional data comprising functional computer data structures to, when loaded into a computer system or network and operated upon thereby, enable said computer system to perform all the steps of the method.

It will be clear to one skilled in the art that many improvements and modifications can be made to the foregoing exemplary embodiments without departing from the scope of the present techniques.

1. A method for delivering an update manifest and an update payload to a target device, the method comprising:
receiving, at the target device, security credentials for the target device, the target device being configured to receive the update manifest and the update payload via a remote connection interface using the security credentials;
receiving, at the target device, the update manifest from a host device via a local connection interface; and
applying, at the target device, the update payload in accordance with the update manifest.

2. A method according to claim 1, wherein the security credentials received at the target device are required to manage the target device via the remote connection interface.

3. A method according to claim 1, wherein the security credentials for the target device comprise a public key and the update manifest is signed with an associated private key.

4. A method according to claim 3, comprising:
verifying, at the target device, the authenticity of the received update manifest by using the public key; and
receiving, at the target device, in response to the verification of the authenticity of the received update manifest, the update payload for the target device via the local connection interface from the host device.

5. A method according to claim 1, wherein the update payload is a firmware update and the update manifest is a firmware manifest.

6. A method according to claim 1, wherein the update manifest comprises a universal asynchronous receiver-transmitter download location for the update payload.

7. A method according to claim 1, comprising:
receiving, at the target device, a first check value from a first cyclic redundancy check on the update payload.

8. A method according to claim 7, wherein the update manifest comprises the first check value from the first cyclic redundancy check.

9. A method according to claim 1, comprising:
receiving the update payload at the target device from the host device via the local connection interface.

10. A method according to claim 9, wherein the update payload received at the target device is stored in a temporary memory of the target device.

11. A method according to claim 7, comprising:
performing, at the target device, a second cyclic redundancy check on the received update payload to compute a second check value; and
comparing the second check value to the first check value, at the target device.

12. A method according to claim 11, wherein if the second check value matches the first check value then a device update client of the target device provides instruction to a target device system bootloader to copy the update payload to a target device flash memory.

13. A method according to claim 12, wherein following the copying of the update payload to the target device flash memory, the target device is restarted.

14. A method according to claim 13, wherein following restart of the target device the security credentials at the target device are maintained.

15. A method according to claim 14, wherein the security credentials comprise identity information for the target device.

16. A method according to claim 1 comprising:
connecting the host device to a mass storage device, the mass storage device being configured to store the update payload and corresponding update manifest for the target device; and
providing the update payload and corresponding update manifest to the host device for sending to the target device.

17. A non-transitory computer readable storage medium comprising code which when implemented on a processor causes the processor to carry out the method of claim 1.

18. A target device comprising:
communication circuitry configured to:
receive security credentials for the target device;
receive an update manifest and an update payload via a remote connection interface using the security credentials; and
receive the update manifest from a host device via a local connection interface; and
a processor configured to:
apply the update payload in accordance with the update manifest.
